SEQUENCE LISTING (24 sequences)

SEQ ID NO: 1 - DNA, 22 bp, Artificial
  Hypothetical sequence
cgccctctacgaactccagtta

SEQ ID NO: 2 - RNA, 19 nt, Artificial
  Hypothetical sequence
cgcccucuacgaacuccag

SEQ ID NO: 3 - RNA, 19 nt, Artificial
  Hypothetical sequence
gcccucuacgaacuccagu

SEQ ID NO: 4 - RNA, 19 nt, Artificial
  Hypothetical sequence
cccucuacgaacuccaguu

SEQ ID NO: 5 - RNA, 19 nt, Artificial
  Hypothetical sequence
ccucuacgaacuccaguua

SEQ ID NO: 6 - DNA, 45 bp, Artificial
  Hypothetical sequence
agctagcacacagactccccccgagaggtctttttccggcatgcc

SEQ ID NO: 7 - DNA, 27 bp, Artificial
  Hypothetical sequence
gcacacagactccccccgagaggtctt

SEQ ID NO: 8 - DNA, 27 bp, Artificial
  Hypothetical sequence
cacacagactccccccgagaggtcttt

SEQ ID NO: 9 - DNA, 27 bp, Artificial
  Hypothetical sequence
acacagactccccccgagaggtctttt

SEQ ID NO: 10 - DNA, 27 bp, Artificial
  Hypothetical sequence
cacagactccccccgagaggtcttttt

SEQ ID NO: 11 - DNA, 27 bp, Artificial
  Hypothetical sequence
acagactccccccgagaggtctttttc

SEQ ID NO: 12 - DNA, 27 bp, Artificial
  Hypothetical sequence
cagactccccccgagaggtctttttcc

SEQ ID NO: 13 - DNA, 27 bp, Artificial
  Hypothetical sequence
agactccccccgagaggtctttttccg

SEQ ID NO: 14 - DNA, 27 bp, Artificial
  Hypothetical sequence
gactccccccgagaggtctttttccgg

SEQ ID NO: 15 - DNA, 27 bp, Artificial
  Hypothetical sequence
actccccccgagaggtctttttccggc

SEQ ID NO: 16 - DNA, 19 bp, Artificial
  Hypothetical sequence
gcacacagactccccccga

SEQ ID NO: 17 - DNA, 19 bp, Artificial
  Hypothetical sequence
cacacagactccccccgag

SEQ ID NO: 18 - DNA, 19 bp, Artificial
  Hypothetical sequence
acacagactccccccgaga

SEQ ID NO: 19 - DNA, 19 bp, Artificial
  Hypothetical sequence
cacagactccccccgagag

SEQ ID NO: 20 - DNA, 19 bp, Artificial
  Hypothetical sequence
acagactccccccgagagg

SEQ ID NO: 21 - DNA, 19 bp, Artificial
  Hypothetical sequence
cagactccccccgagaggt

SEQ ID NO: 22 - DNA, 19 bp, Artificial
  Hypothetical sequence
agactccccccgagaggtc

SEQ ID NO: 23 - DNA, 19 bp, Artificial
  Hypothetical sequence
gactccccccgagaggtct

SEQ ID NO: 24 - DNA, 19 bp, Artificial
  Hypothetical sequence
actccccccgagaggtctt

(19) United States
(12) Patent Application Publication
(10) Pub. No.: US 2006/0160110 A1
(43) Pub. Date: Jul. 20, 2006

(54) Methods of designing small interfering RNAs, antisense polynucleotides, and other hybridizing polynucleotides

(76) Inventors: Takayuki Mizutani, Mountain View, CA (US); Kazuhisa Terasaki, Mountain View, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/290,628

(22) Filed: Nov. 29, 2005

Related U.S. Application Data
(60) Provisional application No. 60/647,193, filed on Jan. 25, 2005.
(60) Provisional application No. 60/632,831, filed on Dec. 2, 2004.

(51) Int. Cl. C12Q 1/68; G06F 19/00

(52) U.S. Cl. 435/6; 702/20

(57) ABSTRACT

The present invention relates to methods, apparatus and computer program products for selecting siRNAs, antisense polynucleotides, and other hybridizing polynucleotides. In particular, the invention relates to methods for selecting siRNAs, antisense polynucleotides, and other hybridizing polynucleotides that have moderate or low off-target activity.

This application claims the benefit of U.S. Provisional Application No. 60/647,193, filed Jan. 25, 2005, and of U.S. Provisional Application No. 60/632,831, filed Dec. 2, 2004. U.S. Provisional Application No. 60/647,193 and U.S. Provisional Application No. 60/632,831 are incorporated herein by reference in their entirety for any purpose.

The present invention relates to methods, apparatus and computer program products for designing small interfering RNAs (siRNAs), antisense polynucleotides, and other hybridizing polynucleotides. The present invention also relates to methods of determining the off-target effects of an siRNA, and antisense polynucleotide, and other hybridizing polynucleotides.

RNA interference is a post-transcriptional process observed in various organisms whereby double-stranded RNA molecules mediate gene silencing in a sequence-specific manner. RNA interference may be carried out using short interfering RNAs (siRNAs), which are generally about 19-22 nucleotides long. Because siRNAs are so short, they may have off-target activity against other identical or similar, but not identical, sequences in a cell. As a result, unintended genes may be silenced by the siRNA.

Antisense polynucleotides may be used to suppress expression of a gene, either before or after transcription. However, an antisense polynucleotide may have off-target activity against other identical or similar, but not identical, sequences in a cell. As a result, unintended genes may be suppressed by the antisense polynucleotide.

Similarly, as with siRNAs and antisense polynucleotides, polynucleotides designed to hybridize with a particular sequence may also have off-target hybridizing activity against other identical or similar, but not identical, sequences in a cell. As a result, the hybridizing polynucleotide may hybridize to unintended sequences.

In certain embodiments, a method for selecting an siRNA of length x is provided. In certain embodiments, the method comprises selecting a target gene, scanning at least a portion of the target gene with a window of size x to identify at least one potential siRNA of length x, performing a sequence analysis on at least one potential siRNA of length x, wherein the sequence analysis comprises assigning a weight value to at least one potential siRNA of length x, and selecting an siRNA of length x from the at least one potential siRNA of length x. In certain embodiments, the sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value. In certain embodiments, the sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

In certain embodiments, a method for identifying predicted off-target genes for an siRNA of length x is provided. In certain embodiments, the method comprises selecting an siRNA of length x, selecting a database, scanning the database with the siRNA of length x to identify one or more potential off-target genes, wherein the potential off-target genes have x/x identical nucleotides or (x-1)/x identical nucleotides or (x-2)/x identical nucleotides to the siRNA of length x, performing a sequence analysis on the one or more potential off-target genes, wherein the sequence analysis comprises assigning an off-target weight value to the one or more potential off-target genes, and identifying predicted off-target genes.

In certain embodiments, a method for selecting an siRNA of length x is provided. In certain embodiments, the method comprises selecting a target gene, scanning at least a portion of the target gene with a window of size x to identify at least one potential siRNA of length x, performing a first sequence analysis on at least one potential siRNA of length x, wherein the sequence analysis comprises assigning a weight value to at least one potential siRNA of length x, selecting a database, scanning the database with at least one potential siRNA of length x to identify one or more potential off-target genes, wherein the potential off-target genes have x/x identical nucleotides or (x-1)/x identical nucleotides or (x-2)/x identical nucleotides to at least one potential siRNA of length x, performing a second sequence analysis on the one or more potential off-target genes, wherein the sequence analysis comprises assigning an off-target weight value to the one or more potential off-target genes, identifying predicted off-target genes for at least one potential siRNA of length x, and selecting an siRNA of length x from the at least one potential siRNA of length x. In certain embodiments, the first sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value. In certain embodiments, the first sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

In certain embodiments, a method of creating a weight table for siRNAs of length x is provided. In certain embodiments, the method comprises making at least two siRNAs of length x to at least one target gene, determining the activity level of each of the at least two siRNAs of length x against the at least one target gene, selecting a threshold activity level, assigning a reduction value of 0 to the threshold activity level, assigning a different positive reduction value to each different activity level greater than the threshold activity level and assigning a different negative reduction value to each different activity level less than the threshold activity level, assigning a reduction value to each siRNA of length x according to its activity level, calculating a weighting factor for adenine (A) in a first position, comprising averaging the reduction value of each siRNA of length x with an adenine (A) in the first position, inserting the weighting factor for adenine (A) in the first position into a weight table, repeating the calculating step and the inserting step for cytosine (C), guanine (G), and uridine (U) in the first position, repeating the calculating step, the inserting step, and the repeating step for at least a second position, thereby creating a weight table for siRNAs of length x. In certain embodiments, the at last two siRNAs of length x are at least 100, at least 200, at least 500, or at least 1000 siRNAs of length x.

In certain embodiments, a method of creating an off-target weight table for siRNAs of length x is provided. In certain embodiments, the method comprises making at least two siRNAs of length x to at least one off-target gene, wherein each siRNA comprises at least one mismatch relative to an off-target gene, determining the adjusted activity level of each of the at least two siRNAs of length x against at least one off-target gene, selecting a threshold adjusted activity level, assigning a reduction value of 0 to the threshold adjusted activity level, assigning a different positive reduction value to each different adjusted activity level greater than the threshold activity level and assigning a different negative reduction value to each different adjusted activity level less than the threshold activity level, assigning a reduction value to each siRNA of length x according to its adjusted activity level, calculating an off-target weighting factor for a mismatch in a first position, comprising averaging the reduction value of each siRNA of length x having a mismatch in the first position, inserting the off-target weighting factor for a mismatch in the first position into a weight table, repeating the calculating step and the inserting step for at least a second position, thereby creating an off-target weight table for siRNAs of length x.

In certain embodiments, a computer program product is provided, comprising a machine readable medium on which is provided program instructions for performing a sequence analysis on at least one potential siRNA of length x. In certain embodiments, a computing device comprising a memory device configured to store at least temporarily program instructions for performing a sequence analysis on at least one potential siRNA of length x. In certain embodiments, the instructions comprise code for scanning at least a portion of a target gene with a window of size x to identify at least one potential siRNA of length x; and code for performing a sequence analysis on at least one potential siRNA of length x, wherein the sequence analysis comprises assigning a weight value to at least one potential siRNA of length x. In certain embodiments, the sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value. In certain embodiments, the sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

In certain embodiments, a computer program product comprises a machine readable medium on which is provided program instructions for performing a sequence analysis on one or more potential off-target genes for an siRNA of length x. In certain embodiments, a computing device comprising a memory device configured to store at least temporarily program instructions for performing a sequence analysis on one or more potential off-target genes for an siRNA of length x. In certain embodiments, the instructions comprise code for scanning a database with an siRNA of length x to identify one or more potential off-target genes, wherein the potential off-target genes have x/x identical nucleotides or (x-1)/x identical nucleotides or (x-2)/x identical nucleotides to the siRNA of length x; and code for performing a sequence analysis on the one or more potential off-target genes, wherein the sequence analysis comprises assigning an off-target weight value to the one or more potential off-target genes.

In certain embodiments, a computer program product comprises a machine readable medium on which is provided program instructions for performing a first sequence analysis on at least one potential siRNA of length x and a second sequence analysis on one or more potential off-target genes for at least one potential siRNA of length x. In certain embodiments, a computing device comprising a memory device configured to store at least temporarily program instructions for performing a first sequence analysis on at least one potential siRNA of length x and a second sequence analysis on one or more potential off-target genes for at least one potential siRNA of length x. In certain embodiments, the instructions comprise code for scanning at least a portion of a target gene with a window of size x to identify at least one potential siRNA of length x; code for performing a first sequence analysis on at least one potential siRNA of length x, wherein the sequence analysis comprises assigning a weight value to at least one potential siRNA of length x; code for scanning a database with at least one potential siRNA of length x to identify one or more potential off-target genes, wherein the potential off-target genes have x/x identical nucleotides or (x-1)/x identical nucleotides or (x-2)/x identical nucleotides to at least one potential siRNA of length x; and code for performing a second sequence analysis on the one or more potential off-target genes, wherein the sequence analysis comprises assigning an off-target weight value to the one or more potential off-target genes. In certain embodiments, the first sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value. In certain embodiments, the first sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value. In certain embodiments, the second sequence analysis further comprises sorting the at least one potential siRNA of length x according to the off-target weight values of the one or more potential off-target genes.

In certain embodiments, a computer program product comprises a machine readable medium on which is provided program instructions for creating a weight table for siRNAs of length x. In certain embodiments, a computing device comprising a memory device configured to store at least temporarily program instructions for creating a weight table for siRNAs of length x. In certain embodiments, the instructions comprise code for assigning a different positive reduction value to each different activity level greater than a selected threshold activity level, code for assigning a different negative reduction value to each different activity level less than the selected threshold activity level; code for assigning a reduction value to an siRNA of length x according to its activity level; code for calculating a weighting factor for adenine (A) in a first, comprising averaging the reduction value of each siRNA of length x with an adenine (A) in the first position; code for inserting the weighting factor for adenine (A) in the first position into a weight table; code for repeating the calculating step and the inserting step for cytosine (C), guanine (G), and uridine (U) in the first position; code for repeating the calculating step, the inserting step, and the repeating step for at least a second position; thereby creating a weight table for siRNAs of length x.

In certain embodiments, a computer program product comprises a machine readable medium on which is provided program instructions for creating an off-target weight table for siRNAs of length x. In certain embodiments, a computing device comprising a memory device configured to store at least temporarily program instructions for creating an off-target weight table for siRNAs of length x. In certain embodiments, the instructions comprise code for assigning a different positive reduction value to each different adjusted activity level greater than a selected threshold activity level; code for assigning a different negative reduction value to each different adjusted activity level less than the selected threshold activity level; code for assigning a reduction value to each siRNA of length x according to its adjusted activity level; code for calculating an off-target weighting factor for a mismatch in a first position, comprising averaging the reduction value of each siRNA of length x having a mismatch in the first position; code for inserting the off-target weighting factor for a mismatch in the first position into a weight table; code for repeating the calculating step and the inserting step for at least a second position; thereby creating an off-target weight table for siRNAs of length x.

In certain embodiments, a method for selecting an siRNA of length y is provided. In certain embodiments, the method comprises selecting a target gene; scanning at least a portion of the target gene with a window of size x to identify at least one potential siRNA of length x, wherein x is less than y; performing a first sequence analysis on at least one potential siRNA of length x, wherein the sequence analysis comprises assigning a weight value to at least one potential siRNA of length x; selecting an siRNA of length x from the at least one potential siRNA of length x; identifying at least one potential siRNA of length y that comprises the siRNA of length x; identifying at least one siRNA of length 19 that is contained within at least one of the at least one potential siRNA of length y; selecting a database; scanning the database with at least one siRNA of length 19 to identify one or more potential off-target genes, wherein the potential off-target genes have 19/19 identical nucleotides or 18/19 identical nucleotides or 17/19 identical nucleotides or 16/19 identical nucleotides to at least one siRNA of length 19; multiplying the number of potential off-target genes having 19/19 identical nucleotides by a first multiplier, multiplying the number of potential off-target genes having 18/19 identical nucleotides by a second multiplier, multiplying the number of potential off-target genes having 17/19 identical nucleotides by a third multiplier, and multiplying the number of potential off-target genes having 16/19 identical nucleotides by a fourth multiplier; determining the predicted off-target effect of each of the at least one siRNAs of length 19; determining the average predicted off-target effect for each of at least one potential siRNA of length y, comprising averaging the predicted off-target effect for all of the at least one siRNAs of length 19 that are contained within each of the at least one potential siRNA of length y; and selecting an siRNA of length y from the at least one potential siRNA of length y. In certain embodiments, the first sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value. In certain embodiments, the first sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

In certain embodiments, a computer program product comprising a machine readable medium on which is provided program instructions is provided. In certain embodiments, the instructions comprise code for scanning at least a portion of the target gene with a window of size x to identify at least one potential siRNA of length x, wherein x is less than y; code for performing a first sequence analysis on at least one potential siRNA of length x, wherein the sequence analysis comprises assigning a weight value to at least one potential siRNA of length x; code for identifying at least one potential siRNA of length y that comprises the siRNA of length x; code for identifying at least one siRNA of length 19 that is contained within at least one of the at least one potential siRNA of length y; code for scanning a database with at least one siRNA of length 19 to identify one or more potential off-target genes, wherein the potential off-target genes have 19/19 identical nucleotides or 18/19 identical nucleotides or 17/19 identical nucleotides or 16/19 identical nucleotides to at least one siRNA of length 19; code for multiplying the number of potential off-target genes having 19/19 identical nucleotides by a first multiplier, multiplying the number of potential off-target genes having 18/19 identical nucleotides by a second multiplier, multiplying the number of potential off-target genes having 17/19 identical nucleotides by a third multiplier, and multiplying the number of potential off-target genes having 16/19 identical nucleotides by a fourth multiplier; code for determining the predicted off-target effect of each of the at least one siRNAs of length 19; and code determining the average predicted off-target effect for each of at least one potential siRNA of length y comprising averaging the predicted off-target effect for all of the at least one siRNAs of length 19 that are contained within each of the at least one potential siRNA of length y. In certain embodiments, the first sequence analysis on at least one potential siRNA of length x further comprises assigning one or more values selected from a G/C value, an end region energy value, an end region A[U value, an end specific energy value, an energy profile value, a melting temperature value, a G/C stretch value, and an additional target value. In certain embodiments, the first sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

In certain embodiments, a computing device comprising a memory device configured to store at least temporarily program instructions is provided. In certain embodiments, the instructions comprise code for scanning at least a portion of the target gene with a window of size x to identify at least one potential siRNA of length x, wherein x is less than y; code for performing a first sequence analysis on at least one potential siRNA of length x, wherein the sequence analysis comprises assigning a weight value to at least one potential siRNA of length x; code for identifying at least one potential siRNA of length y that comprises the siRNA of length x; code for identifying at least one siRNA of length 21 that is contained within at least one of the at least one potential siRNA of length y; code for scanning a database with at least one siRNA of length 19 to identify one or more potential off-target genes, wherein the potential off-target genes have 19/19 identical nucleotides or 18/19 identical nucleotides or 17/19 identical nucleotides or 16/19 identical nucleotides to at least one siRNA of length 19; code for multiplying the number of potential off-target genes having 19/19 identical nucleotides by a first multiplier, multiplying the number of potential off-target genes having 18/19 identical nucleotides by a second multiplier, multiplying the number of potential off-target genes having 17/19 identical nucleotides by a third multiplier, and multiplying the number of potential off-target genes having 16/19 identical nucleotides by a fourth multiplier; code for determining the predicted off-target effect of each of the at least one siRNAs of length 19; and code determining the average predicted off-target effect for each of at least one potential siRNA of length y comprising averaging the predicted off-target effect for all of the at least one siRNAs of length 19 that are contained within each of the at least one potential siRNA of length y. In certain embodiments, the first sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value. In certain embodiments, the first sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

In certain embodiments, a method for selecting an siRNA of length y is provided. In certain embodiments, the method comprises selecting a target gene; scanning at least a portion of the target gene with a window of size x to identify at least one potential siRNA of length x, wherein x is less than y; performing a first sequence analysis on at least one potential siRNA of length x, wherein the sequence analysis comprises assigning a weight value to at least one potential siRNA of length x; selecting an siRNA of length x from the at least one potential siRNA of length x; identifying at least one potential siRNA of length y that comprises the siRNA of length x; identifying at least one siRNA of length 19 that is contained within at least one of the at least one potential siRNA of length y; selecting a database; scanning the database with at least one siRNA of length 19 to identify one or more potential off-target genes, wherein the potential off-target genes have 19/19 identical nucleotides or *18/19 identical nucleotides or 17/19 identical nucleotides or 16/19 identical nucleotides to at least one siRNA of length 19; performing a second sequence analysis on the one or more potential off-target genes comprising assigning an off-target weight value to each of the one or more potential off-target genes; multiplying the sum of the off-target weight values for all of the potential off-target genes having 19/19 identical nucleotides by a first multiplier, multiplying the sum of the off-target: weight values for all of the potential off-target genes having 18/19 identical nucleotides by a second multiplier, multiplying the sum of the off-target weight values for all of the potential off-target genes having 17/19 identical nucleotides by a third multiplier, and multiplying the sum of the off-target weight values for all of the potential off-target genes having 16/19 identical nucleotides by a fourth multiplier; determining the predicted off-target effect of each of the at least one siRNAs of length 19; determining the average predicted off-target effect for each of at least one potential siRNA of length y, comprising averaging the predicted off-target effect for all of the at least one siRNAs of length 19 that are contained within each of the at least one potential siRNA of length y; and selecting an siRNA of length y from the at least one potential siRNA of length y. In certain embodiments, the first sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value. In certain embodiments, the first sequence analysis further comprises sorting at least one potential siRNA of length x according to at least one value selected from a weight, value, a G/C value, an end region energy value, an end region A/U value, an end specific energy value, an energy profile value, a melting temperature value, a G/C stretch value, and an additional target value.

In certain embodiments, a computer program product comprising a machine readable medium on which is provided program instructions is provided. In certain embodiments, the instructions comprise code for scanning at least a portion of the target gene with a window of size x to identify at least one potential siRNA of length x, wherein x is less than y; code for performing a first sequence analysis on at least one potential siRNA of length x, wherein the sequence analysis comprises assigning a weight value to at least one potential siRNA of length x; code for identifying at least one potential siRNA of length y that comprises the siRNA of length x; code for identifying at least one siRNA of length 19 that is contained within at least one of the at least one potential siRNA of length y; code for scanning the database with at least one siRNA of length 19 to identify one or more potential off-target genes, wherein the potential off-target genes have 19/19 identical nucleotides or 18/19 identical nucleotides or 17/19 identical nucleotides or 16/19 identical nucleotides to at least one siRNA of length 19; code for performing a second sequence analysis on the one or more potential off-target genes comprising assigning an off-target weight value to each of the one or more potential off-target genes; code for multiplying the sum of the off-target weight values for all of the potential off-target genes having 19/19 identical nucleotides by a first multiplier; multiplying the sum of the off-target weight values for all of the potential off-target genes-having 18/19 identical nucleotides by a second multiplier, multiplying the sum of the off-target weight values for all of the potential-off-target genes having 17/19 identical nucleotides by a third multiplier, and multiplying the sum of the off-target weight values for all of the potential off-target genes having 16/19 identical nucleotides by a fourth multiplier; code for determining the predicted off-target effect of each of the at least one siRNAs of length 19; and code determining the average predicted off-target effect for each of at least one potential siRNA of length y comprising averaging the predicted off-target effect for all of the at least one siRNAs of length 19 that are contained within each of the at least one potential siRNA of length y. In certain embodiments, the first sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value. In certain embodiments, the first sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

In certain embodiments, a computing device comprising a memory device configured to store at least temporarily program instructions is provided. In certain embodiments, the instructions comprise code for scanning at least a portion of the target gene with a window of size x to identify at least one potential siRNA of length x, wherein x is less than y; code for performing a first sequence analysis on at least one potential siRNA of length x, wherein the sequence analysis comprises assigning a weight value to at least one potential siRNA of length x; code for identifying at least one potential siRNA of length y that comprises the siRNA of length x; code for identifying at least one siRNA of length 21 that is contained within at least one of the at least one potential siRNA of length y; code for scanning the database with at least one siRNA of length 19 to identify one or more potential off-target genes, wherein the potential off-target genes have 19/19 identical nucleotides or 18/19 identical nucleotides or 17/19 identical nucleotides or 16/19 identical nucleotides to at least one siRNA of length 19; code for performing a second sequence analysis on the one or more potential off-target genes comprising assigning an off-target weight value to each of the one or more potential off-target genes; code for multiplying the sum of the-off-target weight values for all of the potential off-target genes having 19/19 identical nucleotides by a first multiplier, multiplying the sum of the off-target weight values for all of the potential off-target genes having 18/19 identical nucleotides by a second multiplier, multiplying the sum of the off-target weight values for all of the potential off-target genes having 17/19 identical nucleotides by a third multiplier, and multiplying the sum of the off-target weight values for-all of the potential off-target genes having 16/19 identical nucleotides by a fourth multiplier; code for determining the predicted off-target effect of each of the at least one siRNAs of length 19; and code determining the average predicted off-target effect for each of at least one potential siRNA of length y comprising averaging the predicted off-target effect for all of the at least one siRNAs of length 19 that are contained within each of the at least one potential siRNA of length y. In certain embodiments, the first sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value. In certain embodiments, the first sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

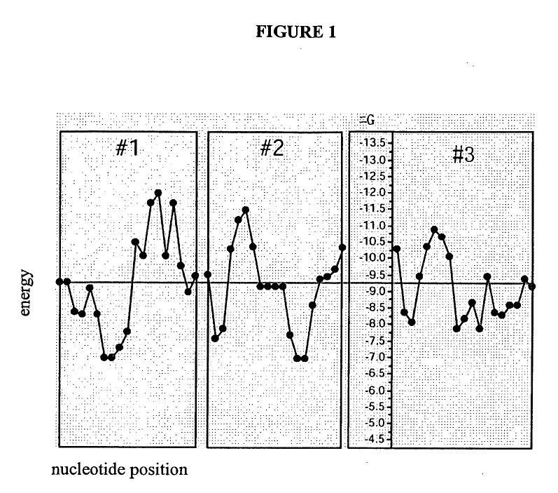

FIG. 1 shows three internal (free) energy profiles for hypothetical siRNAs #1, #2, and #3. Each of those siRNAs comprises 19 base pairs. The x-axis of each graph is the nucleotide position of the siRNA and the y-axis is the internal (free) energy. For example, for siRNA #1, nucleotide positions 1 and 2 each have an internal (free) energy of about −9.25 and nucleotide position 3 has an internal (free) energy of about −8.25.

It is to be understood that this invention is not limited to particular methods, devices or systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise.

Unless defined otherwise, all technical and scientific terms used herein have the same meaning as commonly understood by one of ordinary skill in the art to which the invention pertains. Methods and materials similar or equivalent to those described herein can be used in the practice for testing of the present invention. In describing and claiming the present invention, the following terminology will be used in accordance with the definitions set out below.

As used herein, “siRNA” refers to a double-stranded, RNA molecule that comprises between 12 and 100 nucleotides in each strand. The term “siRNA” includes double-stranded RNAs that comprises two separate RNA molecules, and double-stranded RNAs that comprise a single RNA molecule.

In certain embodiments, one or both ends of an siRNA is blunt-ended, i.e., does not have an overhang. In certain embodiments, an siRNA comprises one or more overhangs. An overhang, as used herein, is a sequence comprising one or more terminal nucleotides that are not base-paired, i.e., are single-stranded. Overhangs may be 5′ overhangs or 3′ overhangs. 5′ overhangs are sequences of 5′ terminal nucleotides that are not base-paired. 3′ overhangs are sequences of 3′ terminal nucleotides that are not base-paired. In certain embodiments, an siRNA comprises one 5′ overhang. In certain embodiments, an siRNA comprises two 5′ overhangs. In certain embodiments, an siRNA comprises one 3′ overhang. In certain embodiments, an siRNA comprises two 3′ overhangs. In certain embodiments, an siRNA comprises one 5′ overhang and one 3′ overhang. In certain embodiments, an overhang comprises 1, 2, 3, 4, or 5 nucleotides. In certain embodiments, an overhang comprises more than 5 nucleotides.

An siRNA comprises two strands. Each siRNA comprises a sense strand and an antisense strand. As used herein, the sense strand of the siRNA does not include nucleotides that are part of an overhang. As used herein, the antisense strand of the siRNA does not include nucleotides that are part of an overhang. In certain embodiments, a strand of siRNA comprises the sense strand and any overhang on a sense strand terminus. In certain embodiments, a strand of siRNA comprises the antisense strand and any overhang on an antisense strand terminus.

In an siRNA that comprises a single RNA molecule, sometimes referred to as a hairpin siRNA, the number of nucleotides in the sense strand or the antisense strand is determined by the number of nucleotides in the strand that are base-paired with nucleotides in the opposite strand. It will be appreciated that not all bases in a strand will necessarily be base-paired and that bulges, overhangs, or mismatches may occur. Thus, the number of nucleotides in one strand of a hairpin siRNA does not include the nucleotides in the single-stranded linker portion of the hairpin siRNA. As a result, as used herein, the sum of the nucleotides in the two strands of a hairpin siRNA may be equal to or less than the total number of nucleotides in the single RNA molecule that forms the hairpin siRNA (because the single-stranded RNA molecule may include one or more nucleotides that are part of a single-stranded linker portion). In certain embodiments, a strand of a hairpin siRNA comprises the sense strand of the hairpin siRNA and any overhang on the terminus of the sense strand that is not the loop of the hairpin siRNA. In certain embodiments, a strand of a hairpin siRNA comprises the antisense strand of the hairpin siRNA and any overhang on the terminus of the antisense strand that is not the loop of the hairpin siRNA. Thus, in certain embodiments, the nucleotides in the loop of the hairpin siRNA are not counted in the length of either strand of the hairpin siRNA.

In certain embodiments, one strand of an siRNA contains between 15 and 150 nucleotides. In certain embodiments, one strand of an siRNA contains between 15 and 1000 nucleotides. In certain embodiments, one strand of an siRNA contains between 15 and 50 nucleotides. In certain embodiments, one strand of an siRNA contains between 15 and 30 nucleotides. In certain embodiments, one strand of an siRNA contains between 17 and 30 nucleotides. In certain embodiments, one strand of an siRNA contains 18, 19, 20, 21, 22, 23, 24, 25, 26, 27, 28, 29, or 30 nucleotides. The two strands of an siRNA may contain the same number of nucleotides or may contain different numbers of nucleotides. In certain embodiments, one strand of the siRNA contains 1, 2, 3, 4, or 5 more nucleotides than the other strand of the siRNA.

As used herein, a “hybridizing polynucleotide” refers to a single-stranded molecule comprised of deoxyribonucleotides, ribonucleotides, or both, which has a sequence that is complementary to at least a portion of a selected target gene. In certain embodiments, a hybridizing polynucleotide is 10, 20, 30, 40, 50, 75, 100, 150, 200, 300, or 500 nucleotides long. “Hybridizing polynucleotide” includes “antisense polynucleotides”, which are hybridizing polynucleotides that are capable of suppressing an antisense target gene.

An siRNA or hybridizing polynucleotide may be produced by any method known in the art. Such methods include, but are not limited to, chemical synthesis, expression of an siRNA or hybridizing polynucleotide from an expression plasmid in a cell, and transcription of an siRNA or hybridizing polynucleotide from a DNA molecule in vitro. An siRNA or hybridizing polynucleotide may be introduced into a cell by any method known in the art, including but not limited to, transfection of the siRNA or hybridizing polynucleotide or an expression vector that encodes the siRNA or hybridizing polynucleotide, infection by a virus that expresses the siRNA or hybridizing polynucleotide, integration into a cell genome of a DNA sequence that expresses the siRNA or hybridizing polynucleotide, and microinjection of the siRNA or hybridizing polynucleotide or an expression vector that encodes the siRNA, antisense polynucleotide, or hybridizing polynucleotide. Transfection methods include, but are not limited to, cesium chloride transfection, lipofection, electroporation, and other methods that increase the permeability of the cell membrane. One skilled in the art can select an appropriate method for introducing an siRNA or hybridizing polynucleotide into a cell. One skilled in the art can also select an appropriate expression vector or viral vector for expressing the siRNA or hybridizing polynucleotide in a cell, if such expression is desired.

In certain embodiments, all of the nucleotides in an siRNA or hybridizing polynucleotide are ribonucleotides. In certain embodiments, an siRNA or hybridizing polynucleotide comprises one or more deoxynucleotides. In certain embodiments, an siRNA or hybridizing polynucleotide comprises only deoxynucleotides. In certain embodiments, one or more nucleotides of the sense strand of the siRNA are deoxynucleotides. In certain embodiments, one or more nucleotides of the antisense strand of the siRNA are deoxynucleotides. In certain embodiments, one or more nucleotides of the sense strand and one or more nucleotides of the antisense strand of the siRNA are deoxynucleotides. In certain embodiments, an siRNA may be a DNA:RNA hybrid. See, e.g., Lamberton et al., Molecular Biotechnology, 24: 111-119 (2003). In certain embodiments, an overhang of an siRNA may comprise one or more deoxynucleotides. In certain embodiments, an overhang of an siRNA may comprise one or more non-naturally occurring nucleotides. Exemplary non-naturally occurring nucleotides include, but are not limited to, nucleotides that form peptide nucleic acids (PNA), nucleotides that form bridged nucleic acids (BNA), and nucleotides that form locked nucleic acids (LNA).

In certain embodiments, an siRNA or hybridizing polynucleotide may comprise one or more ribonucleotides derivatives. Ribonucleotide derivatives that may be used include any derivatives that do not substantially interfere with siRNA or antisense polynucleotide activity. A derivative does not “substantially interfere” with siRNA of antisense polynucleotide activity when the derivative has an-activity that is at least 80% of the activity of an. siRNA or antisense-polynucleotide composed of only naturally-occurring ribonucleotides. Such derivatives include, but are not limited to, derivatives that stabilize RNA molecule(s) under certain conditions, derivatives that increase siRNA or antisense polynucleotide activity, and derivatives that allow for more economical production of siRNAs or hybridizing polynucleotides. Certain exemplary derivatives include, but are not limited to, siRNAs or hybridizing polynucleotides having one or more of 2′-amino-butyryl-pyrene-uridine, 2′-amino-cytidine, 2′-amino-uridine, 2′-deoxy-uridine, 2′-fluoro-cytidine, 2′-fluoro-uridine, 2,6-diaminopurine, 2′-amino-cytidine, 2-aminopurine, 4-thio-uridine, 5-amino-allyl-uridine, 5-bromo-uridine, 5-fluoro-cytidine, 5-fluoro-uridine, 5-iodo-uridine, 5-methyl-cytidine, 5-amino-allyl-uridine, deoxy-abasic, inosine, MN, N3-methyl-uridine, pseudouridine, purine ribonucleoside, ribavirin, ribo-thymidine, 5′-amino-C12 (12 carbon linker), 5′-amino-C3 (3-carbon linker), 5′-amino (5-atom linker), 5′-amino-C6 (6-carbon linker), 5′-biotin, 5′-Cy3, 5′-Cy5, 5′-Dabsyl, 5′-fluorescein, 5′-phosphate, 5′-photocleavable biotin, 5′-tetrachloro-fluorescein, 5′-thiol, 3′-amino modifier, 3′-inverted abasic, 3′-inverted deoxythymidine, 3′-puromycin, deoxy-guanosine, dideoxy-cytidine, 3′ biotin, 3′-Cy3, 3′-Cy5, 3′-fluorescein, 3′-LC biotin, 3′-LC LC biotin, 3′-TAMRA, 5′-PEG-40K, 5′-pyrene, 3′-cholesterol, DNP, and/or 5′-TAMRA-hexyl linker. Certain exemplary derivatives also include, but are not limited to, siRNAs having first rN (rA,rU,rG,rC) in one or both strands, siRNAs or hybridizing polynucleotides having a subsequent rN in one or both strands, and siRNAs or hybridizing polynucleotides having an rW (rA,rU) and/or rS (rC,rG) in one or both strands. Certain exemplary derivatives also include, but are not limited to, siRNAs or hybridizing polynucleotides having one or more phosphorothioate linkages, 18 atom spacers (e.g., hexaethylene glycol), 3-carbon linkers, and/or 9 atom spacers.

As used herein, “target gene” refers to an RNA-encoding sequence that contains a sequence that is identical to either the sense or antisense strand of the siRNA. “Target gene” also refers to the RNA encoded by that target gene. “Target gene” also refers to an RNA that contains a sequence that is identical to either the sense or antisense strand of the siRNA, where that RNA is not encoded from a DNA molecule. Thus, “target gene” includes retroviral RNA sequences and other RNA sequences not transcribed from a DNA molecule, for example. As used herein, “target RNA” refers to an RNA, which may or may not be transcribed from an RNA-encoding sequence, that contains the same sequence as either the sense or antisense strand of the siRNA. Thus, “target RNA” is a subset of “target gene.” Target RNA includes retroviral RNA sequences and other RNA sequences not transcribed from a DNA molecule. “Target RNA” also includes mRNA, which has been transcribed from an RNA-encoding DNA sequence. In certain embodiments, a target RNA is the molecule whose degradation may be mediated by an siRNA, resulting in a reduced level of the target RNA. In certain embodiments, a target RNA is a molecule whose expression is suppressed by the siRNA by any mechanism other than degradation of the target RNA. In certain embodiments, a target gene may be directly suppressed by the siRNA.

In certain embodiments, an “antisense target gene” refers to a sequence, either DNA or RNA, that contains a sequence that is complementary to the sequence of an antisense polynucleotide. In certain embodiments, an antisense polynucleotide reduces the transcription of RNA from an antisense target gene that encodes the RNA. In certain embodiments, an antisense polynucleotide reduces the expression of a protein from an antisense target RNA that encodes the protein.

As used herein, the “activity” or “activity level” of an siRNA refers to the ability of the siRNA to reduce the level of target RNA in a cell. As used herein, “high activity” refers to a reduction in target RNA of 80% or more as determined by quantitative PCR (qPCR) assay. As used herein, “moderate activity” refers to a reduction in target RNA of 50% to 80% as determined by qPCR. As used herein, “⁶low activity” refers to a reduction in target RNA of less than 50% as determined by qPCR. qPCR is described, e.g., in Biotechnology (N Y). April 1992;10(4):413-7; Biotechnology (N Y). September 1993;11(9):1026-30; Methods. December 2001;25(4):402-8; and Gene. Sep. 1, 1990;93(1): 125-8. Other methods of determining siRNA activity are known in the art, including but not limited to, detection of protein expression (including, but not limited to, detection of a marker protein such as GFP or luciferase), detection of RNA using Northern blots, and detection of RNA or cDNA levels using microarrays, detection using bDNA, detection using molecular beacons, and detection using fluorescent oligo probes.

As used herein, the “antisense activity” or “activity level” of an antisense polynucleotide refers to the ability of an antisense polynucleotide to reduce the level of RNA transcribed from an antisense target gene in a cell or to reduce the level of protein expressed from an antisense target RNA in a cell. Methods of determining the level of RNA are known in the art as discussed above. Methods of determining the level of protein are also known in the art and include, e.g., western blotting, HPLC, thin-layer chromatography, ninhydrine and other staining techniques, Bradford assay, etc.

As used herein, “potential off-target gene” refers to a gene that contains a sequence that is similar, but not identical, to either the sense or antisense strand of the siRNA on the coding strand of the gene and within the gene's transcribed region. In certain embodiments, the sequence of the potential off-target gene contains 1, 2, 3, or 4 mismatches as compared to the sense or antisense strand of the siRNA. As used herein, “potential off-target RNA” refers to an mRNA that is transcribed from the potential off-target gene and that contains a sequence that is similar, but not identical, to either the sense or antisense strand of the siRNA.

As used herein, “potential antisense off-target gene” refers to a DNA or RNA that contains a sequence whose complement is similar, but not identical, to the antisense polynucleotide. In certain embodiments, the complement of the potential-antisense off-target gene contains 1%, 2%, 5%, 10%, 15%, 20%, 25%, 30%, 40%, or 50% mismatches relative to the antisense polynucleotide.

As used herein, “similar, but not identical, sequence” refers to a sequence that is at least 80% identical to a reference sequence. In certain embodiments, a sequence is “similar, but not identical” to a reference sequence if it is at least 85%, or at least 90%, or at least 95% identical, to the reference sequence. In certain embodiments, a sequence is “similar, but not identical” to a reference sequence if it is at least 50%, or at least 60%, or at least 75% identical, to the reference sequence. In certain embodiments, if the reference sequence is 19 nucleotides long, a sequence is similar, but not identical, to the reference sequence if the sequence has 11/19, 12/19, 13/19, 14/19, 15/19, 16/19, 17/19, or 18/19 identical nucleotides. In certain embodiments, if the reference sequence is. 19 nucleotides long, a sequence is similar, but not identical, to the reference sequence if the sequence has 11/19, 12/19, 13/19, 14/19, or 15/19 identical nucleotides.

As used herein, “predicted off-target gene” refers to a subset of potential off-target genes. Predicted off-target genes are those genes against which a particular siRNA is predicted, based on any number of selected factors, to have off-target activity. As used herein, “potential off-target RNA” refers to an mRNA that is transcribed from the potential off-target gene and that contains a sequence that is similar, but not identical, to either the sense or antisense strand of the siRNA. Thus, an siRNA is predicted, on average, to have greater off-target activity against a predicted off-target RNA than against a potential off-target RNA that is not a predicted off-target RNA.

As used herein, “predicted antisense off-target gene” refers to a subset of potential antisense off-target genes. Predicted antisense off-target genes are those genes, either RNA or DNA, against which a particular antisense polynucleotide is predicted, based on any number of selected factors, to have off-target antisense activity. Thus, an antisense polynucleotide is predicted, on average, to have greater off-target antisense activity against a predicted antisense off-target gene than against a potential antisense off-target gene that is not a predicted antisense off-target gene.

As used herein, the “off-target activity” of an siRNA refers to the ability of the siRNA to reduce the level of a potential or predicted off-target RNA in a cell. As used herein, “high off-target activity” refers to a reduction in one or more off-target RNAs of 80% or more as determined by quantitative PCR (qPCR) assay. As used herein, “moderate off-target activity” refers to a reduction-in one or more off-target RNAs of 35% to 80% as determined by qPCR. As used herein, “low off-target activity” refers to a reduction in one or more off-target RNAs of less than 35% as determined by qPCR.

As used herein, the “off-target antisense activity” of an antisense polynucleotide refers to the ability of the antisense polynucleotide to reduce the level of a potential or predicted antisense off-target gene in a cell.

As used herein, the. “active strand” of an siRNA refers to the strand of the siRNA that has a sequence that is identical or similar, but not identical, to the target RNA or off-target RNA. In certain embodiments, one strand of an siRNA may be active against a first target gene, while the other strand of an siRNA may be active against a second target gene (or a second region of the first target gene). Thus, one strand of an siRNA may be the active strand with respect to a first target gene, while the other strand may be the active strand with respect to a second target gene.

The present invention provides methods for designing siRNAs and hybridizing polynucleotides, including antisense polynucleotides, that have activity against one or more target genes. The method comprises, in certain embodiments, selecting one or more siRNAs or antisense polynucleotides that are predicted to have activity against a target gene or antisense target gene and then predicting the off-target activity or antisense off-target activity of the one or more selected siRNAs or antisense polynucleotides against potential off-target genes or potential antisense off-target genes.

In certain embodiments, to select one or more siRNAs or antisense polynucleotides that are predicted to have activity against one or more target genes or antisense target genes, factors such as the percentage of Gs and Cs in the siRNA or antisense polynucleotide, the specific nucleotide at each position, the free energy difference between the 3′ and 5′ end regions of an siRNA strand or antisense polynucleotide, the number of A and U nucleotides at the 3′ and 5′ end regions of an siRNA or antisense polynucleotide, the specific energy at the 3′ and 5′ ends of an siRNA strand or antisense polynucleotide, the free energy balance across the siRNA or antisense polynucleotide, the melting temperature of the siRNA or antisense polynucleotide, and the combination of nucleotides in the siRNA or antisense polynucleotide, are considered. In certain embodiments, similar considerations can be used to select any hybridizing polynucleotide for a desired use.

In certain embodiments, the off-target activity or off-target antisense activity of the selected siRNAs or antisense polynucleotides are predicted by scanning a database with the sense and/or antisense strands of the selected siRNAs or with the antisense polynucleotides to identify sequences that are similar, but not identical, to the selected siRNAs or antisense polynucleotides. In certain embodiments, the number and location of mismatches between the potential off-target genes or potential antisense off-target genes and the selected siRNAs or antisense polynucleotides are considered to identify predicted off-target genes or predicted antisense off-target genes.

Sequence Analysis System

In certain instances, randomly selected siRNAs or antisense polynucleotides have variable levels of activity against the target sequence. Thus, certain randomly selected siRNAs or antisense polynucleotides may have low activity against the target sequence, some randomly selected siRNAs or antisense polynucleotides may have moderate activity against the target sequence, and some randomly selected siRNAs or antisense polynucleotides may have high activity against the target sequence.

For simplicity, the Sequence Analysis System is described in the context of selecting an siRNA. Similar methods may be used to select an antisense polynucleotide or any other hybridizing polynucleotide. One skilled in the art can adapt the described methods to uses involving antisense or other hybridizing polynucleotides.

In certain embodiments, a Sequence Analysis System is used to select siRNAs that have a greater likelihood of having moderate or high activity against a particular target gene or genes. In certain embodiments, a Sequence Analysis System is used to select siRNAs that have a greater likelihood of having high activity against a particular target gene or genes. The Sequence Analysis System comprises a collection of criteria that are applied to each siRNA to select siRNAs that have a greater likelihood of having the desired level of activity. Certain criteria in the collection of criteria produce a “value” that is assigned to the siRNA or off-target gene. Certain criteria in the collection of criteria produce a “value” that is assigned to one or more nucleotides of the siRNA or off-target gene. The value in either case need not be a number. In certain embodiments, a value may be a binary indication of state, i.e., the presence or absence of something. The Sequence Analysis System may comprise some or all of the criteria discussed herein, as well as additional criteria selected by the user. In certain embodiments, by altering the Sequence Analysis System criteria and thresholds, one skilled in the art can select one or more siRNAs that have a desired threshold level of activity.

To use the Sequence Analysis System, in certain embodiments, a desired target gene or genes is selected. A desired target gene may be a portion of an RNA-encoding sequence or a portion of an RNA. In certain embodiments, siRNAs that have activity against more than one target gene can be selected by choosing a target gene region that is identical, or similar, but not identical, or has regions of identity, among the selected target genes. One skilled in the art can choose such a target gene region, e.g., by aligning the sequences and finding a region or regions that are identical or similar, but not identical, or have regions of identity among the sequences. One or more of the regions can then be used in the Sequence Analysis System (for simplicity, a region that is used in the Sequence Analysis System will be referred to as a “target gene”, even though that region represents a portion of a coding sequence and/or represents multiple coding sequences). If a region contains differences between the selected target genes, siRNAs that target sequences containing those differences can be eliminated from the pool of potential siRNAs.

In certain embodiments, once a target gene is selected, the target gene is scanned with a fixed-size window to identify one or more possible siRNAs of that fixed size against the target sequence. In certain embodiments, the target gene is scanned with a fixed-size window to identify all possible siRNAs of that fized size against the target sequence. For example, for the sequence:

| | |
|---|---|
| 5′ CGCCCTCTACGAACTCCAGTTA 3′ | [SEQ ID NO:. 1] |

all possible siRNAs of fixed size 19 nucleotides are shown below:

| | |
|---|---|
| 5′ CGCCCUCUACGAACUCCAG 3′ | [SEQ ID NO: 2] |
| 5′ GCCCUCUACGAACUCCAGU 3′ | [SEQ ID NO: 3] |
| 5′ CCCUCUACGAACUCCAGUU 3′ | [SEQ ID NO: 4] |
| 5′ CCUCUACGAACUCCAGUUA 3′ | [SEQ ID NO: 5] |

Thus, in certain embodiments, given a target gene of length N nucleotides and a window fixed at size X nucleotides, there will be (N−X+1) possible siRNAs of fixed size X to that target gene. That calculation assumes that each siRNA has a second strand that is completely base paired to the first strand and that there are no non-base paired overhangs on either strand. Of course, one skilled in the art could alter the second strand to create varying lengths of overhangs and increase the total number of possible siRNAs to a target gene. In certain embodiments, the window is fixed at between 15 and 150 nucleotides. In certain embodiments, the window is fixed at between 15 and 100 nucleotides. In certain embodiments, the window is fixed at between 15 and 50 nucleotides. In certain embodiments, the window is fixed at 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 39, or 50 nucleotides.

In certain embodiments, the Sequence Analysis System comprises one or more of the following criteria. In certain embodiments, the Sequence Analysis System selects, on average, higher activity siRNAs when more criteria are used to select the siRNAs.

G/C Criteria

In certain embodiments, the Sequence Analysis System applies a G/C criteria. The G/C criteria considers the G/C base pair content of the possible siRNAs and produces a G/C value. In certain embodiments, a G/C value is expressed as a percentage. To consider the G/C base pair content, in certain embodiments, the G/C criteria calculates the percentage of G/C base pairs relative to total base pairs of an siRNA. Thus, in certain embodiments, non-base paired nucleotides are not considered when calculating the G/C base pair content of an siRNA. For example, for the four possible siRNAs discussed above, if none of the siRNAs contain non-base paired nucleotides, then the G/C contents of SEQ ID NOs: 2-5 are 63.2%, 57.9%, 52.6%, and 47.4%, respectively.

In certain embodiments, the G/C criteria favors siRNAs that have a G/C content of between 30% and 65%. In certain embodiments, the G/C criteria favors siRNAs that have a G/C content of between 35% and 60%. In certain embodiments, the G/C criteria favors siRNAs that have a G/C content of between 40% and 55%. In certain embodiments, siRNAs that have a G/C content outside of the chosen range are not considered further. In certain embodiments, siRNAs that have a G/C content outside of the chosen range are retained in the list of possible siRNAs, but siRNAs that satisfy the G/C criteria are grouped or otherwise ordered or identified (such as, for example, by including an indication of the percentage of G/C base pairs for each siRNA) to distinguish them from siRNAs that do not satisfy the G/C criteria.

Weight Value Criteria

In certain embodiments, the Sequence Analysis System applies a Weight Value criteria. The Weight Value criteria considers the identity of the particular nucleotide at each position of an siRNA and produces a weight value. In certain embodiments, the Weight Value criteria applies a weight table to each siRNA. The weight table assigns a weighting factor to each possible nucleotide at each position. Thus, after applying the weight table to an siRNA, a particular weighting factor is applied to each position of the siRNA depending on which nucleotide is at that position. In certain embodiments, the Sequence Analysis System adds together all of the weighting factors for a particular siRNA to arrive at a weight value for that siRNA.

In certain embodiments, a method of creating a weight table is as follows. A series of siRNAs of equal length are made against one or more target genes. The activity level of each siRNA against its target gene is determined. In certain embodiments, the activity level is calculated as a percentage reduction in target RNA as determined by qPCR. In certain embodiments, a threshold activity level is assigned a reduction value of 0, and all activity levels above that threshold are assigned positive reduction values and all activity levels below that threshold are assigned negative reduction values.

In certain embodiments, each nucleotide in an siRNA is assigned the siRNA reduction value (thus, each nucleotide is assigned the same reduction value). A weighting factor based on the reduction value is calculated for one or more of the nucleotides at various positions of the siRNA. In certain embodiments, a weighting factor is calculated for each of the four nucleotides at various positions of the siRNA. In certain embodiments, a weighting factor is calculated for each of the four nucleotides at each of the positions of the siRNA. In certain embodiments, the weighting factor is a statistical measure of the varabilitiy in the reduction values. In certain embodiments, the weighting factor can be, for example, an average, a mean, or other statistical measure.

In certain embodiments, a weight table is compiled using data from at least 50 siRNAs to one or more target genes. In certain embodiments, a weight table is compiled using data from at least 100 siRNAs to one or more target genes. In certain embodiments, a weight table is compiled using data from at least 150 siRNAs to one or more target genes. In certain embodiments, a weight table is compiled using data from at least 300 siRNAs to one or more target genes. In certain embodiments, a weight table is compiled using data from at least 500 siRNAs to one or more target genes. In certain embodiments, a weight table is compiled using data from at least 750 siRNAs to one or more target genes. In certain embodiments, a weight table is compiled using data from at least 1000 siRNAs to one or more target genes. In certain embodiments, a weight table is compiled using data from at least 2000 siRNAs to one or more target genes. In certain embodiments, a weight table is compiled using data from at least 5000 siRNAs to one or more target genes. In certain embodiments, as additional siRNAs are tested for activity, the new data is added to the weight table calculation to refine the weight table.

In certain embodiments, the Weight Value criteria applies a weight table to each siRNA to assign a weighting factor to each position of the siRNA. In certain embodiments, the Weight Value criteria then sums the weighting factors to arrive at a total weight value for each siRNA. The siRNAs may then be ordered according to total weight value. In certain embodiments, siRNAs with total weight values below a certain threshold are removed from consideration.

End Region Energy Criteria

In certain embodiments, the Sequence Analysis System applies a End Region Energy criteria. The End Region Energy criteria considers the free energy difference between the 5′ region and the 3′ region of the siRNA. The End Region Energy criteria produces an end region energy value. In certain embodiments, the 5′ region of the siRNA is defined as the first 3, 4, 5, 6, or 7 nucleotides of the sense strand of the siRNA. In certain embodiments, the 5′ region of the siRNA is defined as the first 5 nucleotides of the sense strand of the siRNA. In certain embodiments, the 3′ region of the siRNA is defined as the last 3, 4, 5, 6, or 7 nucleotides of the sense strand of the siRNA. In certain embodiments, the 3′ region of the siRNA is defined as the last 5 nucleotides of the sense strand of the siRNA. In certain embodiments, the free energy of the 5′ region and the 3′ region is determined using the method described, e.g., in Cell. Oct. 17, 2003;115(2):209-16 (erratum in: Cell. Nov. 14, 2003; 115(4):505); and Cell. Oct. 17, 2003;115(2):199-208. In certain embodiments, the End Region Energy criteria favors siRNAs that have lower free energy in the 3′ region than in the 5′ region. In certain embodiments, the end region energy value may comprise a value for each of the 5′ region and the 3′ region. In certain embodiments, the end region energy value may comprise a single value that takes into account the energy of the both the 3′ and 5′ regions. That single value may either be related to the actual energy of those regions or a binary indication of whether the 3′ or 5′ region has higher energy.

End Region A/U Criteria

In certain embodiments, rather than, or in addition to, applying the End Region Energy criteria, the Sequence Analysis System applies the End Region A/U criteria. The End Region A/U criteria considers the number of A/U base pairs in the 5′ region of the siRNA and in the 3′ region of the siRNA and produces an end region A/U value. In certain embodiments, the End Region A/U criteria prefers siRNAs with a higher number of A/U base pairs in the 3′ region versus the 5′ region. In certain embodiments, the end region A/U value may comprise a value for each of the 5′ region and the 3′ region. In certain embodiments, the end region A/U value may comprise a single value that takes into account the number of A/U base pairs in the both the 3′ and 5′ regions, e.g., by subtracting one number from the other. In certain embodiments, the end region A/U value may be a binary indication of whether the 3′ or 5′ region has more A/U base pairs.

End Specific Energy Criteria

In certain embodiments, the Sequence Analysis System applies an End Specific Energy criteria. The End Specific Energy criteria considers the specific energy at the 5′ end and at the 3′ end of the sense strand of each siRNA and produces an end specific energy value. In certain embodiments, the End Specific, Energy criteria considers the specific energy of the first nucleotide and the last nucleotide of the sense strand of each siRNA. In certain embodiments, the End Specific Energy criteria calculates the specific energy using a method known in the art, e.g., as described in Cell. Oct. 17, 2003;115(2):209-16 (erratum in: Cell. Nov. 14, 2003; 115(4):505); and Cell. Oct. 17, 2003;115(2):199-208. In certain embodiments, the End Specific Energy criteria prefers siRNAs with a lower specific energy at the last nucleotide than at the first nucleotide. In certain embodiments, the end specific energy value may comprise a value for each of the 5′ end and the 3′ end. In certain embodiments, the end specific energy value may comprise a single value that takes into account the energy of the both the 3′ and 5′ ends. That single value may either be related to the actual energy of those ends or a binary indication of whether the 3′ or 5′ end has higher specific energy.

Energy Profile Criteria

In certain embodiments, the Sequence Analysis System applies an Energy Profile criteria. The Energy Profile criteria considers the internal (free) energy at each position of each siRNA and produces an energy profile value. In certain embodiments, Energy Profile criteria calculates the internal (free) across the siRNA according to a method known in the art, e.g., as described in Proc Natl Acad Sci USA. December 1986;83(24):9373-7; and Cell. Oct. 17, 2003;115(2):209-16 (erratum in: Cell. Nov. 14, 2003;115(4):505). In certain embodiments, the Energy Profile criteria prefers siRNAs in which the internal (free) energy of each position is between −7 kcal/mol and −11 kcal/mol. In certain embodiments, narrower energy profiles are favored over broader energy profiles. In certain embodiments, siRNAs with energy profiles that are similar to the energy profiles of one or more known high activity siRNAs are favored. For example, if three siRNAs, numbered 1, 2, and 3, have the internal (free) energy profiles shown in FIG. 1, siRNA 3 is preferred. When siRNAs 1, 2, and 3 were tested for activity against their target genes, siRNA 3 reduced target RNA by 98%, while siRNAs 1 and 2 reduced target RNA by 16% and 96%, respectively. A negative control siRNA, which has a scrambled sequence relative to the target gene, had an activity level of 1 0%.

In certain embodiments, the energy profile value may comprise a value for each of nucleotides of the sequence. In certain embodiments, the energy profile value may comprise two values, one indicating the highest energy of the sequence and one indicating the lowest energy of the sequence. In certain embodiments, the energy profile value may be a binary indication of whether the sequence exceeds the preferred energy range or not.

Melting Temperature Criteria

In certain embodiments, the Sequence Analysis System applies a Melting Temperature criteria. The Melting Temperature criteria considers the melting temperature of each siRNA and produces a melting temperature value. In certain embodiments, the Melting Temperature criteria calculates the melting temperature of the siRNA by any method known in the art, for example, according to the method, described, e.g., in Nat Biotechnol. March 2004;22(3):326-30. In certain embodiments, the Melting Temperature criteria prefers siRNAs with lower melting temperatures to siRNAs with higher melting temperatures. In certain embodiments, the melting temperature value is expressed as the melting temperature of the sequence.

G/C Stretch Criteria

In certain embodiments, the Sequence Analysis System applies a G/C Stretch criteria. The G/C Stretch criteria considers whether each siRNA comprises a stretch of consecutive G and C nucleotides and produces a G/C stretch value. In certain embodiments, the G/C Stretch criteria prefers siRNAs that so not contain any stretches of 4 or more consecutive G and/or C nucleotides. For example, siRNAs containing the sequences AGGCGT, ACCCCA, TGGCGCA, etc., each have a stretch of 4 or more consecutive G and/or C nucleotides and are not preferred. In certain embodiments, the G/C stretch value is expressed as the highest number of consecutive G and/or C nucleotides in a sequence. In certain embodiments, the G/C stretch value is expressed as a binary indication of whether the sequence contains a stretch of 4 or more consecutive G and/or C nucleotides or not.

Criteria Weighting Factor

In certain embodiments, one or more criteria used in the Sequence Analysis System are weighted according to a criteria weighting factor, such that some criteria are considered more important than others in selecting siRNAs. In certain embodiments, the criteria weighting factor is determined empirically. In certain embodiments, a collection of siRNAs are tested for activity. Any number of siRNAs can be tested to determine criteria weighting factors. The number of siRNAs having high activity, low moderate activity, and low activity are identified. A criteria described herein is then applied and those siRNAs that fall within such criteria are identified. The relative amount of siRNAs having high activity and falling within the critiera are calculated. Similarly, the relative amount of siRNAs having low activity and falling within the criteria are calculated. Comparison of those relative amounts yields a weighting factor for such criteria.

In certain embodiments, a criteria weighting factor is determined for each criteria used in the Sequence Analysis System.

In certain embodiments, application of the Sequence Analysis System results in a list of possible siRNAs that are ranked according to their predicted activity against the target gene. However, the activity of the siRNAs may not correspond to their rank in the Sequence Analysis System. In certain embodiments, the average activity of the top 10% of ranked siRNAs is greater than the average activity of the bottom 10% of ranked siRNAs. In certain embodiments, the average activity of the top 20% of ranked siRNAs is greater than the.average activity of the bottom 20% of ranked siRNAs. In certain embodiments, a subset of the ranked siRNAs are selected for further analysis. In certain embodiments, at least 5, 10, 20, 30, 50, 75, or 100 of the ranked siRNAs are selected for further analysis. In certain embodiments, all of the ranked siRNAs are subject to further analysis.

Additional Target Criteria

In certain embodiments, the Sequence Analysis System applies an Additional Target criteria. In certain embodiments, an Additional Target criteria is applied after the other criteria in the Sequence Analysis System. In certain embodiments, when the Additional Target criteria is applied after the other criteria of the Sequence Analysis System, the Additional Target criteria is applied to a subset of siRNAs. In certain embodiments, the Additional Target criteria is applied to all of the siRNAs.

In certain embodiments, all or a subset of siRNAs are scanned against a selected database to identify identical or similar, but not identical, sequences to each siRNA to produce an additional target value. The sense and/or antisense strand of the siRNA may be scanned against the selected database. To scan an siRNA against a selected database, the siRNA of length X is compared to each sequence of length X in the database. Thus, similar to the window used to identify all possible siRNAs, the known siRNA sequence defines the window and is used to scan the database. If a sequence in the database is identical or similar, but not identical, to the siRNA sequence, the gene containing that sequence is flagged, along with the number of nucleotide matches. For example, an siRNA having 19 nucleotides may be scanned against a selected database with a threshold identity level of 17/19 nucleotides. In this example, scanning reveals two genes that have sequences with 19/19 identical nucleotides, seven genes that have sequences with 18/19 identical nucleotides, and 40 genes that have sequences with 17/19 identical nucleotides.

Potential databases include, but are not limited to, species-specific databases, cDNA databases, genomic databases, databases containing SNPs; databases containing splice variants, tissue-specific databases, developmental stage-specific databases, mRNA databases, and protein databases, and databases containing a combination of any of the above. Thus, for example, a selected database may be an embryonic human brain cDNA database containing all known splice variants and SNPs. Such a database would contain cDNA sequences corresponding to known RNAs and known splice variants expressed in human embryonic brains. The database would also contain all known SNPs in those particular RNAs and known splice variants. One skilled in the art can select the appropriate database for a particular use.

In certain embodiments, each strand of each siRNA is compared to the selected database to identify identical sequences. If an identical sequence is found in a gene other than the original selected target gene, then the siRNA may have activity against that second gene as well. A second gene having a sequence identical to the sense or antisense strand of the siRNA is considered a target gene, even though that gene was not the originally selected target gene. In certain embodiments, if it is not desirable to reduce the expression of the second target gene along with the expression of the first target gene, then the siRNA is removed from the list of possible siRNAs or the siRNA is otherwise not considered further.

In certain embodiments, each stand of each siRNA is compared to the selected database to identify sequences that are similar, but not identical. In certain embodiments, the number of sequences in the database that have a particular identity to each siRNA is determined. For example, the number of sequences in the database that have (X-1)/X (e.g., 18/19, 19/20, etc.) identical nucleotides or (X-2)/X identical nucleotides, etc., is determined. In certain embodiments, siRNAs for which there are sequences in the database that have (X-1)/X identical nucleotides are removed from the list of possible siRNAs or are not otherwise considered further. In certain embodiments, siRNAs for which there are sequences in the database that have (X-1)/X identical nucleotides are tested against those sequences to determine if there are off-target effects against those sequences. In certain embodiments, if off-target effects are found, those siRNAs are removed from the list or are otherwise not considered further.

In certain embodiments, an additional target value indicates the number of identical sequences to the siRNA found in the selected database. In certain embodiments, the additional target value also indicates the number of similar, but not identical, sequences to the siRNA found in the selected database, with a separate number for each level of identity, i.e., the number of sequences that have (X-1)/X nucleotides identical to the siRNA, the number of sequences that have (X-2)/X nucleotides to the siRNA, etc. In certain embodiments, the additional target value is a binary indication of whether or not there are additional sequences in the selected database, outside of the original target gene, that are identical to the siRNA.

Off-Target Prediction System

In certain embodiments, the off-target activity of an siRNA or antisense polynucleotide is predicted using an Off-Target Prediction System. In certain embodiments, the off-target activity or off-target antisense activity of an siRNA or antisense polynucleotide is predicted by identifying potential off-target genes or potential antisense off-target genes. In certain embodiments, from the potential off-target genes or potential antisense off-target genes, predicted off-target genes or predicted antisense off-target genes are identified. In certain embodiments, the off-target activity or off-target antisense activity of all or a portion of the siRNAs or antisense polynucleotides identified in the Sequence Analysis System is determined.

For simplicity, the Off-Target Prediction System is described in the context of siRNAs. Similar methods may be used for antisense polynucleotides or any other hybridizing polynucleotides. One skilled in the art can adapt the described methods to uses involving antisense or other hybridizing polynucleotides.

In certain embodiments, a database is selected, as discussed above. Thus, for example, if the siRNA is intended to be used in a specific tissue, a tissue-specific database can be selected to predict the off-target effects. Alternatively, the off-target effects can be predicted for any siRNA using, e.g., a complete species-specific database. One skilled in the art can select the appropriate database for analyzing of-target effects according to the intended use for the siRNA.

In certain embodiments, each strand of each siRNA is compared to the selected database as discussed above, by scanning the database with the sequence, to identify sequences that are similar, but not identical, which are potential off-target genes. In certain embodiments, the potential off-target genes that have a particular identity to each siRNA is determined. In certain embodiments, the potential off-target genes that have (X-1)/X identical nucleotides to the siRNA are identified. In certain embodiments, the potential off-target genes that have (X-2)/X identical nucleotides to the siRNA are identified.

Once the potential off-target genes are identified, the potential off-target genes that fall into each group (i.e., potential off-target genes having (X-1)/X identical nucleotides, off-target genes having (X-2)/X identical nucleotides, etc.) are analyzed using the Off-Target Prediction System to identify predicted off-target genes.

In certain embodiments, the Off-Target Prediction System comprises one or more of the following criteria. In certain embodiments, the Off-Target Prediction System, on average, more accurately identifies predicted off-target genes when more criteria are used in the system.

Off-Target Weight Value Criteria

In certain embodiments, the Off-Target Prediction System applies an Off-Target Weight Value criteria. The Off-Target Weight Value criteria considers the location of the one or more mismatches between the siRNA being analyzed and the potential off-target gene. In certain embodiments, the Off-Target Weight Value criteria applies an off-target weight table to the siRNA or to the potential off-target gene.

In certain embodiments, an off-target weight table may be created as follows. In certain embodiments, the general effect of mismatches in various location of an siRNA relative to an off-target gene is determined. A series of siRNAs to one or more target genes is made in which each siRNA is similar, but not identical to, the siRNA relative to the target gene. The off-target activity level of each siRNA against the target gene is determined by methods known in the art. In certain embodiments, the off-target activity level of each siRNA is expressed as a percent reduction in target RNA as determined, e.g, by qPCR. The percent reduction can be adjusted to arrive at an adjusted percent reduction (or adjusted activity level).

In certain embodiments, a threshold adjusted activity level is assigned a reduction value of 0, and all adjusted activity levels above that threshold are assigned positive reduction values and all adjusted activity levels below that threshold are assigned negative reduction values. For example, 50% adjusted reduction in target RNA (i.e., 50% adjusted activity level) is assigned a reduction value of 0, 1060% adjusted reduction (i.e., 100% adjusted activity level) is assigned a reduction value of 100, and 0% adjusted reduction (i.e., 0% adjusted activity level) is assigned a reduction value of −100. Thus, in that example, if an off-target siRNA reduces target RNA by 90% relative to the fully-matched siRNA (i.e., has a 90% adjusted activity level), the off-target siRNA is assigned a reduction value of 80 and if an off-target siRNA reduces target RNA by 40% relative to the fully-matched siRNA (i.e., has a 40% adjusted activity level), the off-target siRNA is assigned a reduction value of −20.

In certain embodiments; the siRNAs with mismatches are sorted according to the number of mismatches relative to the target gene. In certain embodiments, the location of the mismatch is then identified in each siRNA and each mismatch is assigned the reduction value of the siRNA. The reduction values for all mismatches in one location are then added and then divided by the number of siRNAs having mismatches in that location to produce a weighting factor. As a result, if mismatches in that location result in less activity of the siRNAs, then the weighting factor for that location will be low or negative. Mismatches in that location are therefore predicted to result in less off-target activity of the siRNA. Those locations, in turn, are considered to be conserved regions. Alternatively, if mismatches in a particular location have little effect on the activity of the siRNA, then the weighting factor for that location will be a high number. That location is predicted to be a less conserved region because mismatches have little effect on activity.

In certain embodiments, an off-target weight table may be created as follows. In certain embodiments, target gene sequence or an siRNA that is identical to the target gene sequence is selected (if a target gene sequence is selected, it has the same length as the siRNA that would be selected). The following steps can be carried out with either the selected target gene sequence or the selected siRNA, but for convenience, the steps will be discussed as though an siRNA were selected. In certain embodiments, the sense strand of the siRNA is used to scan a selected database to identify identical target gene sequences and similar, but not identical, off-target gene sequences. In certain embodiments, if the siRNA has 19 nucleotides, the database is scanned to identify sequences that have 12 or more, for example, 14 or more, such as 16 or more identical nucleotides. In certain embodiments, the antisense strand of the siRNA is also used to scan the database. In certain embodiments, after identifying potential off-target genes and additional target genes, the activity of the siRNA against one or more of the potential off-target genes and/or additional off-target genes is determined. The off-target activity may be determined, in various embodiments, using qPCR, detection of protein expression (including, but not limited to, detection of a marker protein such as GFP or luciferase), detection of RNA using Northern blots, and detection of RNA or cDNA levels using microarrays, and detection using bDNA. In certain embodiments, the off-target activity is adjusted to arrive at an adjusted activity level.

In certain embodiments, the off-target weight table includes weighting factors for matches at each position of the siRNA in addition to weighting factors for mismatches at each position of the siRNA. In certain embodiments, the weighting factors for matches at each position of the siRNA are calculated in the same manner as the weighting factor for mismatches, except the reduction values of each siRNA having a match in that position are added and then divided by the number of siRNAs having matches at that location to produce a weighting factor.

In certain embodiments, a threshold adjusted activity level is assigned a reduction value of 0, and all adjusted activity levels above that threshold are assigned positive reduction values and all adjusted activity levels below that threshold are assigned negative reduction values. For example, 50% adjusted activity level is assigned a reduction value of 0, 100% adjusted activity level is assigned a reduction value of 100, and 0% adjusted activity level is assigned a reduction value of −100. Thus, in that example, if an siRNA has 90% activity against an off-target gene relative to its activity against the selected target gene, the siRNA is assigned a reduction value of 80 for that off-target gene. It can also be said that that off-target gene has a reduction value of 80 for that siRNA. Further, if an siRNA has 40% for a second off-target gene relative to its activity against the selected target gene, the siRNA is assigned a reduction value of −20 for that second off-target gene. It can also be said that the second off-target gene has a reduction value of −20 for that siRNA.

In certain embodiments, the off-target genes with mismatches relative to the selected siRNA are sorted according to the number of mismatches. In certain embodiments, the location of the mismatch is then identified in each off-target gene and each mismatch is assigned the reduction value of the off-target gene. The reduction values for all mismatches in one location are added together and then divided by the number of off-target genes having a mismatch in that location relative to the selected siRNA to produce a weighting factor. As a result, if mismatches in that location result in less activity by the selected siRNA for the off-target genes having mismatches in that location, then the weighting factor for that location will be low or negative. Mismatches in that location are therefore predicted to result in less off-target activity of the siRNA. Those locations, in turn, are considered to be conserved regions. Alternatively, if mismatches in a particular location have little effect on the activity of the selected siRNA for off-target genes having mismatches in that location, then the weighting factor for that location will be a high number. That location is predicted to be a less conserved region because mismatches have little effect on activity.

In certain embodiments, mismatches in an siRNA are additive. Thus, if an siRNA with a mismatch at position A shows a 20% reduction in activity relative. to an siRNA with no mismatches, and an siRNA with a mismatch at position B shows a 10% reduction in activity relative to an siRNA with no mismatches, then an siRNA with mismatches at positions A and B is expected to show a 30% reduction in activity relative to an siRNA with no mismatches.

In certain embodiments, the Off-Target Weight Value criteria applies an off-target weight table that includes weighting factors for matches at one or more positions of the siRNA in addition to weight values for mismatches at one or more positions of the siRNA. In certain embodiments, the following method can be used to arrive at weighting factors for matches at each position. A collection of siRNAs that are similar, but not identical, to an off-target gene are tested for activity against the off-target gene. The siRNAs are grouped according to high activity, moderate activity, and low activity. The siRNAs are then grouped according to the matching nucleotide at each position and the percent of siRNAs with that matching nucleotide having high, moderate, and low off-target activity is used to arrive at a weighting factor for a match of that nucleotide at that position.

In certain embodiments, combinations of multiple mismatches may be given weighting factors in a similar way.

The Off-Target Weight Value criteria applies the off-target weight table to each of the potential off-target genes identified by the database scan of the selected siRNA. The off-target weight table assigns match and mismatch off-target weighting factors to each position of the potential off-target gene according to whether that position is a match or a mismatch with respect to the selected siRNA. In certain embodiments, the Off-Target Weight Value criteria sums the off-target weighting factors to arrive at an off-target weight value for each potential off-target gene.

In certain embodiments, the potential off-target genes are then sorted according to the off-target weight value. In certain embodiments, a higher off-target weight value indicates that the selected siRNA is predicted to have higher activity against that potential off-target gene. The potential off-target genes against which the selected siRNA is predicted to have higher activity are referred to as predicted off-target genes. In certain embodiments, the potential off-target genes that have off-target weight values above a certain threshold are considered predicted off-target genes.

In certain embodiments, mismatch position is used to predict off-target acitivity. In certain embodiments, a 19mer siRNA that has mismatches in one or more of positions in a first segment of the potential off-target gene is predicted to have less off-target activity against that potential off-target gene as compared to other off-target genes with the same number of mismatches elsewhere.

In certain embodiments, the Sequence Analysis System and/or the Off-Target Prediction System can be altered to take into account the results of additional siRNA or antisense polynucleotide experiments. Thus, each type of criteria may be adjusted as additional data is collected. Similarly, weight tables may be adjusted as additional data on siRNA or antisense polynucleotide activity and off-target activity is collected. Thus, the Sequence Analysis System and Off-Target Prediction System can be improved over time as more data is used to compile weight tables and adjust criteria. In certain embodiments, the Sequence Analysis System and the Off-Target Prediction Systems become more accurate as the criteria are adjusted to accommodate more data.

In certain embodiments, the Sequence Analysis System and Off-Target Prediction System can be used to select siRNAs having 17, 18, 19, 20, 21, 22, 23, 24, 25, 26, 27, 28, 29, or 30 base pairs. An exemplary method of selecting an siRNA of length y using a Sequence Analysis System that selects siRNAs of length x (where x is less than y), with each siRNA of length y having at least z nucleotides in common with the siRNA of length x (where z is equal to or less than x), is as follows. The Sequence Analysis System is used to select siRNAs of length x that have a greater likelihood of having moderate or high activity against a particular target gene or genes, as discussed above. For each siRNA of length x, all possible siRNAs of length y that have at least z nucleotides in common with the siRNA of length x are identified. The off-target effect of each 21 mer that can be made from each siRNA of length y, is then determined. The off-target effect of all of the 21mers that can be made from each siRNA of length y is then averaged, and the siRNA of length y having the lowest average off-target effect is selected.

For example, one can select an siRNA of length 27 using a Sequence Analysis System that selects siRNAs of length 19, and with the requirement that each siRNA of length 27 contain all 19 nucleotides in common with the siRNA of length 19, as follows. First, the Sequence Analysis System is used to select 19mer siRNAs that have a greater likelihood of having moderate or high activity against a particular target gene or genes, as discussed above. For each 19mer selected, every possible 27mer that contains all 19 nucleotides of that 19mer is determined. For each 19mer, therefore, there are 9 possible 27mers, as illustrated for a 19mer having the following sequence (shown in capital letters) and having the following surrounding sequence (shown in lower case letters):

(SEQ ID NO: 6)

agctagcacacagACTCCCCCCGAGAGGTCTTtttccggcatgcc

All possible 27mers containing 19 nucleotides in common with the 19mer are:

| | | |
|---|---|---|
| 1. | gcacacagACTCCCCCCGAGAGGTCTT | (SEQ ID NO: 7) |
| 2. | cacacagACTCCCCCCGAGAGGTCTTt | (SEQ ID NO: 8) |
| 3. | acacagACTCCCCCCGAGAGGTCTTtt | (SEQ ID NO: 9) |
| 4. | cacagACTCCCCCCGAGAGGTCTTtt | (SEQ ID NO: 10) |
| 5. | acagACTCCCCCCGAGAGGTCTTtttc | (SEQ ID NO: 11) |
| 6. | cagACTCCCCCCGAGAGGTCTTtttcc | (SEQ ID NO: 12) |
| 7. | agACTCCCCCCGAGAGGTCTTtttccg | (SEQ ID NO: 13) |
| 8. | gACTCCCCCCGAGAGGTCTTtttccgg | (SEQ ID NO: 14) |
| 9. | ACTCCCCCCGAGAGGTCTTtttccggc. | (SEQ ID NO: 15) |

For each 27mer, all possible 19mers contained within that 27mer are then identified. For example, for 27mer number 1, above, the following 19mers are identified:

| | | |
|---|---|---|
| 1A. | gcacacagACTCCCCCCGA | (SEQ ID NO: 16) |
| 1B. | cacacagACTCCCCCCGAG | (SEQ ID NO: 17) |
| 1C. | acacagACTCCCCCCGAGA | (SEQ ID NO: 18) |
| 1D. | cacagACTCCCCCCGAGAG | (SEQ ID NO: 19) |
| 1E. | acagACTCCCCCCGAGAGG | (SEQ ID NO: 20) |
| 1F. | cagACTCCCCCCGAGAGGT | (SEQ ID NO: 21) |
| 1G. | agACTCCCCCCGAGAGGTC | (SEQ ID NO: 22) |
| 1H. | gACTCCCCCCGAGAGGTCT | (SEQ ID NO: 23) |
| 1I. | ACTCCCCCCGAGAGGTCTT. | (SEQ ID NO: 24) |

The predicted off-target effect of each of those 19mers is then determined using the Off-Target Prediction System. For example, for 19mer 1A, the number of off-target genes having 19/19 identical nucleotides, 18/19 identical nucleotides, 17/19 identical nucleotides, or 16/19 identical nucleotides to either strand of l9mer 1A is determined. The number of off-target genes having 19/19,identical nucleotides is then multiplied by an off-target multiplier of, e.g., 1.0. The number of off-target genes having 18/19 identical nucleotides is then multiplied by an off-target multiplier of, e.g., 0.9. The number of off-target genes having 17/19 identical nucleotides is then multiplied by an off-target multiplier of, e.g., 0.8. The number of off-target genes having 16/19 identical nucleotides is then multiplied by an off-target multiplier of, e.g., 0.6. The predicted off-target effect of 19mer 1A is the sum of the number of off-target genes having each level of identity multiplied by the appropriate off-target multiplier. Thus, for example, if 19mer 1A has 1 off-target gene with 19/19 identical nucleotides, 3 off-target genes with 18/19 identical nucleotides, 5 off-target genes with 17/19 identical nucleotides, and 27 off-target genes with 16/19 identical nucleotides, then the predicted off-target effect is [(1×10)+(3×0.9)+(5×0.8)+(27×0.6)], which is 23.9.

One skilled in the art can select appropriate multipliers for each of level of identity between an off-target genes and a l9mer. In certain embodiments, the off-target weight value for each off-target gene identified for a particular 19mer is considered when calculating a predicted off-target effect. As a non-limiting example when off-target weight values are considered, rather than multiplying the number off off-target genes having a particular percent identity with the off-target multiplier, the sum of the off-target weight values of all of the off-target genes having that percent identity to the selected 19mer can be multiplied by the off-target multiplier.

The predicted off-target effect is averaged across all of the 19mers that correspond to a single 27mer to arrive at an average predicted off-target effect for that 27mer. The process of identifying all 19mers and determining their predicted off-target effect is then repeated for each 27mer. As a result, each 27mer is assigned an average predicted off-target effect. In certain embodiments, the 27mer with the lowest average predicted off-target effect is selected.

In certain embodiments, when selecting a 27mer using a Sequence Analysis System that identifies siRNAs of length 19, a buffer of 1, 2, 3, 4, 5, or 6 nucleotides can be required on one or both ends of the 19mer core. Thus, as a non-limiting example, in the example discussed above, if a 2 nucleotide buffer were required on each end of the 19mer core, then there would be 5 possible 27mers that comprise the selected 19mer. Those 5 would be 27mer numbers 3 through 7, above. As a further non-limiting example, if a 2 nucleotide buffer were required on the 5′ end and a 3 nucleotide buffer were required on the 3′ end, then there would be 4 possible 27mers that comprise the selected 19mer. Those 4 would be 27mer numbers 4 through 7, above.

The foregoing example is not intended to limit the invention in any way. siRNAs of any length, including but not limited to 27, can be selected in the same manner, using a Sequence Analysis System that selects siRNAs of any length. Furthermore, a system can be designed that requires any number of nucleotides in common between the siRNA identified by the Sequence Analysis System and the differently-sized siRNAs identified around that one siRNA, as discussed above. In addition, a system can be designed that requires any number of nucleotides as a buffer on one or both ends of the original siRNA core sequence.

Generally, embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the, computer using an interpreter.

Although the above has generally described the present invention according to specific processes and apparatus, the present invention has a much broader range of applicability. In particular, aspects of the present invention is not limited to any particular kind of cellular process and can be applied to virtually any cellular process where an understanding of the affect of a treatment on a cell is desired. Thus, in some embodiments, the techniques of the present invention could provide information about many different types or groups of cells, substances, cellular processes and mechanisms of action, and genetic processes of all kinds. One of ordinary skill in the art would recognize other variants, modifications and alternatives in light of the foregoing discussion.

Having now fully described the invention, it will be appreciated by those skilled in the art that the invention can be performed within a range of equivalents and conditions without departing from the spirit and scope of the invention and without undue experimentation. In addition, while the invention has been described in light of certain embodiments and examples, the inventors believe that it is capable of further modifications. This application is intended to cover any variations, uses, or adaptations of the invention which follow the general principles set forth above.

The specification includes recitation to the literature and those literature references are herein specifically incorporated by reference. The appendix filed herewith is expressly incorporated herein by reference for any purpose.

The specification and examples are exemplary only with the particulars of the claimed invention set forth as follows:

1. A method for selecting an siRNA of length x, comprising selecting a target gene;
scanning at least a portion of the target gene with a window of size x to identify at least one potential siRNA of length x;
performing a sequence analysis on at least one potential siRNA of length x, wherein the sequence analysis comprises assigning a weight value to at least one potential siRNA of length x;
selecting an siRNA of length x from the at least one potential siRNA of length x.

2. The method of claim 1, wherein the sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value.

3. The method of claim 2, wherein the sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

4. A method for identifying predicted off-target genes for an siRNA of length x, comprising
selecting an siRNA of length x;
selecting a database;
scanning the database with the siRNA of length x to identify one or more potential off-target genes, wherein the potential off-target genes have x/x identical nucleotides or (x-1)/x identical nucleotides or (x-2)/x identical nucleotides to the siRNA of length x;
performing a sequence analysis on the one or more potential off-target genes, wherein the sequence analysis comprises assigning an off-target weight value to the one or more potential off-target genes; and
identifying predicted off-target genes.

5. A method for selecting an siRNA of length x, comprising:
selecting a target gene;
scanning at least a portion of the target gene with a window of size x to identify at least one potential siRNA of length x;
performing a first sequence analysis on at least one potential siRNA of length x, wherein the sequence analysis comprises assigning a weight value to at least one potential siRNA of length x;
selecting a database;
scanning the database with at least one potential siRNA of length x to identify one or more potential off-target genes, wherein the potential off-target genes have x/x identical nucleotides or (x-1)/x identical nucleotides or (x-2)/x identical nucleotides to at least one potential siRNA of length x;
performing a second sequence analysis on the one or more potential off-target genes, wherein the sequence analysis comprises assigning an off-target weight value to the one or more potential off-target genes; and
identifying predicted off-target genes for at least one potential siRNA of length x selecting an siRNA of length x from the at least one potential siRNA of length x.

6. The method of claim 5, wherein the first sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value.

7. The method of claim 6, wherein the first sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

8. A method of creating a weight table for siRNAs of length x comprising making at least two siRNAs of length x to at least one target gene;
determining the activity level of each of the at least two siRNAs of length x against the at least one target gene;
selecting a threshold activity level;
assigning a reduction value of 0 to the threshold activity level;
assigning a different positive reduction value to each different activity level greater than the threshold activity level and assigning a different negative reduction value to each different activity level less than the threshold activity level;
assigning a reduction value to each siRNA of length x according to its activity level;
calculating a weighting factor for adenine (A) in a first position, comprising averaging the reduction value of each siRNA of length x with an adenine (A) in the first position;
inserting the weighting factor for adenine (A) in the first position into a weight table;
repeating the calculating step and the inserting step for cytosine (C), guanine (G), and uridine (U) in the first position;
repeating the calculating step, the inserting step, and the repeating step for at least a second position;
thereby creating a weight table for siRNAs of length x.

9. The method of claim 8 wherein the at least two siRNAs of length x is at least 100 siRNAs of length x.

10. The method of claim 8 wherein the at least two siRNAs of length x is at least 200 siRNAs of length x.

11. The method of claim 8 wherein the at least two siRNAs of length x is at least 500 siRNAs of length x.

12. The method of claim 8 wherein the at least two siRNAs of length x is at least 1000 siRNAs of length x.

13. A method of creating an off-target weight table for siRNAs of length x comprising:
making at least two siRNAs of length x to at least one off-target gene, wherein each siRNA comprises at least one mismatch relative to an off-target gene;
determining the adjusted activity level of each of the at least two siRNAs of length x against at least one off-target gene;
selecting a threshold adjusted activity level;
assigning a reduction value of 0 to the threshold adjusted activity level;
assigning a different positive reduction value to each different adjusted activity level greater than the threshold activity level and assigning a different negative reduction value to each different adjusted activity level less than the threshold activity level;
assigning a reduction value to each siRNA of length x according to its adjusted activity level;
calculating an off-target weighting factor for a mismatch in a first position, comprising averaging the reduction value of each siRNA of length x having a mismatch in the first position;
inserting the off-target weighting factor for a mismatch in the first position into a weight table;
repeating the calculating step and the inserting step for at least a second position;
thereby creating an off-target weight table for siRNAs of length x.

14-33. (canceled)

34. A method for selecting an siRNA of length y, comprising:
selecting a target gene;
scanning at least a portion of the target gene with a window of size x to identify at least one potential siRNA of length x, wherein x is less than y;
performing a first sequence analysis on at least one potential siRNA of length x, wherein the sequence analysis comprises assigning a weight value to at least one potential siRNA of length x;
selecting an siRNA of length x from the at least one potential siRNA of length x;
identifying at least one potential siRNA of length y that comprises the siRNA of length x;
identifying at least one siRNA of length 19 that is contained within at least one of the at least one potential siRNA of length y;
selecting a database;
scanning the database with at least one siRNA of length 19 to identify one or more potential off-target genes, wherein the potential off-target genes have 19/19 identical nucleotides or 18/19 identical nucleotides or 17/19 identical nucleotides or 16/19 identical nucleotides to at least one siRNA of length 19;
multiplying the number of potential off-target genes having 19/19 identical nucleotides by a first multiplier, multiplying the number of potential off-target genes having 18/19 identical nucleotides by a second multiplier, multiplying the number of potential off-target genes having 17/19 identical nucleotides by a third multiplier, and multiplying the number of potential off-target genes having 16/19 identical nucleotides by a fourth multiplier;
determining the predicted off-target effect of each of the at least one siRNAs of length 19;
determining the average predicted off-target effect for each of at least one potential siRNA of length y, comprising averaging the predicted off-target effect for all of the at least one siRNAs of length 19 that are contained within each of the at least one potential siRNA of length y;
selecting an siRNA of length y from the at least one potential siRNA of length y.

35. The method of claim 34, wherein the first sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value.

36. The method of claim 35, wherein the first sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

37-42. (canceled)

43. A method for selecting an siRNA of length y, comprising:
selecting a target gene;
scanning at least a portion of the target gene with a window of size x to identify at least one potential siRNA of length x, wherein x is less than y;
performing a first sequence analysis on at least one potential siRNA of length x, wherein the sequence analysis comprises assigning a weight value to at least one potential siRNA of length x;
selecting an siRNA of length x from the at least one potential siRNA of length x;
identifying at least one potential siRNA of length y that comprises the siRNA of length x;
identifying at least one siRNA of length 19 that is contained within at least one of the at least one potential siRNA of length y;
selecting a database;
scanning the database with at least one siRNA of length 19 to identify one or more potential off-target genes, wherein the potential off-target genes have 19/19 identical nucleotides or 18/19 identical nucleotides or 17/19 identical nucleotides or 16/19 identical nucleotides to at least one siRNA of length 19;
performing a second sequence analysis on the one or more potential off-target genes comprising assigning an off-target weight value to each of the one or more potential off-target genes;
multiplying the sum of the off-target weight values for all of the potential off-target genes having 19/19 identical nucleotides by a first multiplier, multiplying the sum of the off-target weight values for all of the potential off-target genes having 18/19 identical nucleotides by a second multiplier, multiplying the sum of the off-target weight values for all of the potential off-target genes having 17/19 identical nucleotides by a third multiplier, and multiplying the sum of the off-target weight values for all of the potential off-target genes having 16/19 identical nucleotides by a fourth multiplier;
determining the predicted off-target effect of each of the at least one siRNAs of length 19;
determining the average predicted off-target effect for each of at least one potential siRNA of length y, comprising averaging the predicted off-target effect for all of the at least one siRNAs of length 19 that are contained within each of the at least one potential siRNA of length y;
selecting an siRNA of length y from the at least one potential siRNA of length y.

44. The method of claim 43, wherein the first sequence analysis on at least one potential siRNA of length x further comprises assigning one or more values selected from a G/C value, an end region energy value, an end region A/U value, an end specific energy value, an energy profile value, a melting temperature value, a G/C stretch value, and an additional target value.

45. The method of claim 44, wherein the first sequence analysis further comprises sorting at least one potential siRNA of length x according to at least one value selected from a weight value, a G/C value, an end region energy value, an end region A/U value, an end specific energy value, an energy profile value, a melting temperature value, a G/C stretch value, and an additional target value.

46-51. (canceled)
